(19) United States
(12) Patent Application Publication
(10) Pub. No.: US 2024/0373894 A1
(43) Pub. Date: Nov. 14, 2024

(54) SLEEP INDUCTION AND SLEEP QUALITY IMPROVEMENT DRINK

(71) Applicant: MUSKA CO.,LTD, Seoul (KR)

(72) Inventor: Eun Kyung KIM, Seoul (KR)

(21) Appl. No.: 18/577,986

(22) PCT Filed: Aug. 11, 2022
(86) PCT No.: PCT/KR2022/012031

§ 371 Date: Jan. 9, 2024

(30) Foreign Application Priority Data
Aug. 12, 2021 (KR) 10-2021-0106381
Aug. 11, 2022 (KR) 10-2022-0100269

(51) Int. Cl. A23L 29/281; A23L 27/30; A23L 33/105; A23L 33/15

(52) U.S. Cl. CPC A23L 29/281; A23L 27/30; A23L 33/105; A23L 33/15

(57) ABSTRACT

The present invention relates to a sleep induction or sleep disorder improvement composition comprising natural product-derived ingredients as active ingredients, more specifically to a sleep induction or sleep disorder improvement composition that is capable of comprising natural product-derived ingredients as active ingredients, without having the side effects chemical drugs have, thereby providing excellent sleep induction or sleep disorder improvement. According to the present invention, the sleep induction or sleep disorder improvement composition comprising natural product-derived ingredients as active ingredients decreases the sleep latency and increases the sleep duration so that sleep induction and sleep quality improvement are excellent, and accordingly, the composition of the present invention is useful in sleep induction or sleep disorder improvement.

### TECHNICAL FIELD

The present invention relates to a sleep induction or sleep disorder improvement composition comprising natural product-derived ingredients as active ingredients, more specifically to a sleep induction or sleep disorder improvement composition that is capable of comprising natural product-derived ingredients as active ingredients, without having the side effects chemical drugs have, thereby providing excellent sleep induction or sleep disorder improvement.

### BACKGROUND ART

Sleep disorders are one of problems modern people often have had. The sleep disorders are conditions that do not get normal sleep, fail to be able to stay awake during the day despite getting enough sleep, or disturb normal sleep patterns to cause difficulty in getting sleep or being awakened. The sleep disorders occur along with mental health conditions, such as excessive stress, anxiety, fear, and the like. The sleep disorders are one of the most common clinical problems that occur in up to about 20% of population. The sleep disorders may cause various individual and social problems, such as learning disorders, lowering of efficiency, traffic accidents, safety accidents, emotional disorders, social adjustment disorders, unsatisfaction of marriage life, industrial accidents, and the like. If the sleep disorders are not treated well, medical, neurologic, or mental diseases may become severe or delayed in recovery, and further, serious diseases, such as myocardial infarction, stroke, and the like may occur.

Benzodiazepines and serotonin agonists are used as treatment drugs for sleep disorders. Many drugs such as Benzodiazepines bind to GABAA receptors to perform pharmacological actions, and if drugs or supplements bind to Benzodiazepines, the affinity of the GABAA receptors to GABA and the introduction of chlorine ions into cells become increased, thereby achieving anxiety release, convulsion improvement, relax, and sleep induction and improvement. The GABAA receptors are pentameric proteins making membrane ion channels, which have close relation with adjustment of relax, sleep, anxiety, muscle strain, convulsion, and memory loss, and through the GABAA receptors, GABA (y-aminobutyric acid) works. When such drugs are used for a long period of time, side effects, such as drug resistance, drug dependence, and the like are accompanied to cause serious problems such as muscle relief, forgetfulness, and the like.

Due to such side effects of the drugs, demands for natural sleep aids replacing the drugs have increased by sleeping pill long-term users and persons who are limited in the use of the sleeping pill. In developed countries, markets of health functional foods for sleep improvement are expanded, and with the development of Lifestyle of health and sustainability (LOHAS) trend and the increasing interest in sleep, desires to enhance sleep quality become raised. Accordingly, demands for sleep improvement compositions will increase greatly by common people as well as insomnia patients.

Like this, the interest of modern people in the sleep aids having excellent sleep induction or sleep disorder improvement, without having any side effects, have increased, and therefore, the inventors of the present invention have studied to develop a natural product-derived composition that is safe and has excellent efficacy. Finally, they propose a sleep induction or sleep disorder improvement composition according to the present invention.

### DISCLOSURE

### Technical Problem

It is an object of the present invention to provide a food composition for sleep induction or sleep disorder improvement.

It is another object of the present invention to provide a health functional food composition for sleep induction or sleep disorder improvement.

### Technical Solution

To accomplish the above-mentioned objects, according to one aspect of the present invention, there is provided a food composition for sleep induction or sleep disorder improvement.

To accomplish the above-mentioned objects, according to another aspect of the present invention, there is provided a health functional food composition for sleep induction or sleep disorder improvement.

### Advantageous Effects

According to the present invention, the composition for sleep induction or sleep disorder improvement makes use of a variety of natural materials, thereby minimizing side effects, decreasing sleep latency, and increasing sleep duration.

### DESCRIPTION OF DRAWINGS

FIG. 1 is a list showing the Pittsburgh sleep quality index.

FIG. 2 shows a sleep quality evaluation (primary evaluation) result for one month before a sleep induction or sleep disorder improvement composition comprising natural product-derived ingredients as active ingredients according to the present invention is taken.

FIG. 3 shows a sleep quality evaluation (secondary evaluation) result for a week during which the sleep induction or sleep disorder improvement composition comprising natural product-derived ingredients as active ingredients according to the present invention is taken, after the primary evaluation with the Pittsburgh sleep quality index has been completed.

FIG. 4 shows a sleep quality evaluation result for a week after a week, after taking the sleep induction or sleep disorder improvement composition comprising natural product-derived ingredients as active ingredients according to the present invention has been completed after the completion of the secondary evaluation with the Pittsburgh sleep quality index.

### BEST MODE FOR INVENTION

A sleep induction or sleep disorder improvement composition comprising natural product-derived ingredients as active ingredients according to the present invention is capable of decreasing sleep latency and increasing sleep duration.

### MODE FOR INVENTION

The terms used in the present invention are to be given their ordinary and customary meaning to a person of ordinary skill in the art, and are not to be limited to a special or customized meaning unless expressly so defined herein. It should be noted that the use of particular terminology when describing certain features or aspect of the disclosure should not be taken to imply that the terminology is being re-defined herein to be restricted to include any specific characteristics of the features or aspects of the disclosure with which that terminology is associated. Therefore, persons skilled in the relevant art can appreciate that many modifications and variations are possible in light of the above teachings. It is therefore intended that the scope of the invention be limited not by this detailed description, but rather by the claims appended hereto.

The terms used in the present invention are to be given their ordinary and customary meaning to a person of ordinary skill in the art, and are not to be limited to a special or customized meaning unless expressly so defined herein. It should be noted that the use of particular terminology when describing certain features or aspect of the disclosure should not be taken to imply that the terminology is being re-defined herein to be restricted to include any specific characteristics of the features or aspects of the disclosure with which that terminology is associated.

In the description, when it is said that one portion is described as “includes” any component, one element further may include other components unless no specific description is suggested.

The present invention may be modified in various ways and may have several exemplary embodiments. Specific exemplary embodiments of the present invention are illustrated in the drawings and described in detail in the detailed description. However, this does not limit the invention within specific embodiments and it should be understood that the invention covers all the modifications, equivalents, and replacements within the idea and technical scope of the invention.

The present invention provides a food composition or health functional food composition for sleep induction or sleep disorder improvement.

The present invention provides a composition for sleep induction or sleep disorder improvement.

In detail, the composition for sleep induction or sleep disorder improvement according to the present invention may comprise 15 to 25 parts by weight of Siberian ginseng trunk, 10 to 20 parts by weight of wheat, 10 to 20 parts by weight of lotus seed, 10 to 20 parts by weight of jujube, 5 to 15 parts by weight of jujube seed, 5 to 15 parts by weight of gardenia, 5 to 15 parts by weight of Poria cocos, 0.5 to 5 parts by weight of red ginseng, and 1 to 10 parts by weight of Ecklonia cava extract powder in the total weight thereof as 100 parts by weight, more desirably may comprise 18 to 22 parts by weight of Siberian ginseng trunk, 12 to 16 parts by weight of wheat, 12 to 16 parts by weight of lotus seed, 12 to 16 parts by weight of jujube, 6 to 10 parts by weight of jujube seed, 6 to 10 parts by weight of gardenia, 6 to 10 parts by weight of Poria cocos, 1 to 4 parts by weight of red ginseng, and 5 to 7 parts by weight of Ecklonia cava extract powder in the total weight thereof as 100 parts by weight, and if the respective amounts of the above-mentioned materials are greater than or less than the set ranges, it is hard to expect the sleep induction or sleep disorder improvement.

Further, the composition of the present invention may comprise one or more active ingredients selected from the group consisting of Siberian ginseng trunk, wheat, lotus seed, jujube, jujube seed, gardenia, Poria cocos, red ginseng, and Ecklonia cava extract powder as natural products, and in addition thereto, the composition of the present invention may comprise one or more ingredients selected from the group consisting of tart cherry juice powder, fish collagen, beet root concentrate powder, vitamin C, enzymatically modified stevia, and L-theanine.

In this case, the composition of the present invention may comprise 0.5 to 3 parts by weight of Siberian ginseng trunk, 0.5 to 2.5 parts by weight of wheat, 0.5 to 2.5 parts by weight of lotus seed, 0.5 to 2.5 parts by weight of jujube, 0.1 to 1.5 parts by weight of jujube seed, 0.1 to 1.5 parts by weight of gardenia, 0.1 to 1.5 parts by weight of Poria cocos, 0.05 to 0.25 parts by weight of red ginseng, 0.1 to 0.8 parts by weight of Ecklonia cava extract powder, 0.5 to 5 parts by weight of tart cherry juice powder, 0.1 to 0.5 parts by weight of fish collagen, 0.1 to 0.5 parts by weight of beet root concentrate powder, 0.1 to 0.6 parts by weight of vitamin C, 0.1 to 0.6 parts by weight of enzymatically modified stevia, and 0.01 to 0.15 parts by weight of L-theanine in the total weight thereof as 100 parts by weight, more desirably may comprise 1 to 2 parts by weight of Siberian ginseng trunk, 0.8 to 1.5 parts by weight of wheat, 0.8 to 1.5 parts by weight of lotus seed, 0.8 to 1.5 parts by weight of jujube, 0.4 to 0.8 parts by weight of jujube seed, 0.4 to 0.8 parts by weight of gardenia, 0.4 to 0.8 parts by weight of Poria cocos, 0.1 to 0.25 parts by weight of red ginseng, 0.3 to 0.6 parts by weight of Ecklonia cava extract powder, 1 to 4 parts by weight of tart cherry juice powder, 0.15 to 0.4 parts by weight of fish collagen, 0.1 to 0.3 parts by weight of beet root concentrate powder, 0.1 to 0.5 parts by weight of vitamin C, 0.1 to 0.4 parts by weight of enzymatically modified stevia, and 0.05 to 0.10 parts by weight of L-theanine in the total weight thereof as 100 parts by weight, and if the respective amounts of the above-mentioned materials are greater than or less than the set ranges, it is hard to expect the sleep induction or sleep disorder improvement.

In the description, the term “extract” represents a substance that is extracted from the natural product, and the extract may include an extracted liquid, a diluted or concentrated liquid of the extracted liquid, a dried substance obtained by drying the extracted liquid, a roughly purified substance or purified product thereof, and a fraction thereof.

In the description, the term “sleep induction or sleep disorder improvement” represents sleep latency decreasing or sleep duration increasing.

In the description, the term “sleep disorder” represents all of diseases related to sleep, which have disorders in sleep due to various reasons. In detail, sleep disorders occur when a person does not get sufficient amount of sleep or deep sleep, so that he or she has chronic fatigue, feels drowsy in daytime, or has a headache, thereby causing many problems in his or her daily life, and further, sleep disorders occur when he or she does not sleep easily or does not get deep sleep even though he or she goes to sleep. In this case, deep sleep is a state where a person sleeps deeply for 4 to 6 hours, without having any movement, to a degree where he or she does not dream or does not remember whether how much he or she sleep.

The sleep disorders are one or more disorders selected from the group consisting of sleep latency disorder, deep sleep disorder, awakening on night sleep, early morning awakening, insomnia, nightmare, sleepwalking, narcolepsy, unusual behaviors during sleep, hypersomnia, sleep seizure, sleep-disordered breathing, sleep apnea, circadian rhythm sleep disorder, parasomnia, restless legs syndrome, and periodic limb movement disorder, but they may not be limited thereto.

In the description, the term “improvement” represents all types of behaviors for reducing parameters related to relief or treatment of a condition, for example, degrees of symptoms.

According to the present invention, if the composition of the present invention is taken, sleep latency decreases and sleep duration increases, thereby improving sleep quality remarkably. Accordingly, it can be appreciated that the composition of the present invention has efficacies of sleep induction promotion and sleep disorder improvement.

In the description, the term “composition” generally represents a substance that is in a condition directly edible through a given treatment of natural products or processed products having one or more nutrients, and types of compositions may not be limited particularly. The types of compositions may include compositions (e.g., fruits, vegetable compositions, soy milk, fermented compositions, ice cream, etc.), vitamin complex, alcohol compositions, alcoholic liquors, and other liquids, but they may not be limited thereto. Food additives of the composition may be made through typical manufacturing methods.

In the description, food generally means a natural or processed product having one or more nutrients, desirably a natural or processed product that becomes in a condition directly edible through a given treatment, and types of food may not be limited particularly. The food composition may include food, food additives, health functional food, and drink.

For example, the food to which the composition of the present invention is added includes all types of food, drinks, gum, candies, tea, vitamin complex, functional food, etc. In addition, the food of the present invention includes foodstuffs intended for particular nutritional uses (e.g., milk formula, infant/baby food, etc.), processed meat products, fish products, tofu, jelly, noodles (e.g., ramen, noodle soup, etc.), health supplement food, seasonings (e.g., soy sauce, soybean paste, hot pepper paste, mixed paste, etc.), sources, snack, processed milk products (e.g., fermented milk, cheese, etc.), other processed products, Kimchi, pickled foods (all types of Kimchi, pickled vegetables, etc.), drinks (e.g., fruit drinks, vegetable drinks, soy milk, fermented drinks, ice cream, etc.), natural condiment (e.g., ramen soup base), vitamin complex, alcoholic drinks, alcoholic liquors, and other health supplement food, but the food of the present invention may not be limited thereto. The food, drinks, or food additives may be made through typical manufacturing methods.

According to the present invention, if the composition of the present invention is taken, mean sleep induction time is 6.7 minutes, and mean sleep duration is 5.8 hours. Accordingly, it can be checked that the sleep latency becomes decreased and the sleep duration becomes increased (See Experimental example 1).

According to the present invention, the Pittsburgh sleep quality indexes are evaluated for a month before the composition of the present invention is taken, after taking the composition for a week, and for a week after the taking is stopped, respectively, and their respective result values are 12.29, 6.25, and 7, which shows that the evaluation values after taking are about two times lower than those before taking, and accordingly, it can be appreciated that the composition of the present invention improves sleep quality remarkably (See Experimental example 2).

According to the present invention, it can be checked that the composition of the present invention in which tart cherry juice powder is contained has a high overall preference score in sensory evaluation (See Experimental example 3).

Accordingly, it is obvious that the composition of the present invention and the food having the composition have remarkable sleep quality and sleep disorder improvement and excellent preference improvement.

Hereinafter, embodiments of the present invention will be explained in detail.

Further, claims of the present invention are not limited by the following embodiments, but it is intended that the claims be interpreted to embrace all such variations and modifications.

### Embodiment 1. Manufacturing a Composition for Sleep Induction or Sleep Disorder Improvement

### 1-1. Preparation of a Concentrate Mixture Before Mixing

Ingredients for making a mixture as will be mentioned below were purchased on the market.

70 kg (combination percentage of 20.649% (w/w)) of Eleutherococcus sessiliflorus (domestically produced), 50 kg (combination percentage of 14.749% (w/w)) of wheat (domestically produced), 50 kg (combination percentage of 14.749% (w/w)) of lotus seed (produced in Vietnam), 50 kg (combination percentage of 14.749% (w/w)) of jujube (domestically produced), 30 kg (combination percentage of 8.850% (w/w)) of jujube seed (produced in Myanmar), 30 kg (combination percentage of 8.850% (w/w)) of gardenia (produced in China), 30 kg (combination percentage of 8.850% (w/w)) of Poria cocos (produced in China), 8 kg (combination percentage of 2.360% (w/w)) of red ginseng (domestically produced), and 21 kg (combination percentage of 6.195% (w/w)) of Ecklonia cava extract powder (domestically produced) (See Table 1). The Ecklonia cava extract powder represents the powder of Ecklonia cava extract.

To perform the mixing, further, a mixing tank (Daegu Technopark biocentre) and a pipe were pre-sterilized with water heated to 95° C. or more for 5 minutes or more.

TABLE 1

| | Combination | % by |
|---|---|---|
| | amount(kg) | weight (w/w) |
| Siberian ginseng trunk | 70 | 20.649 |
| Wheat | 50 | 14.749 |
| Lotus seed | 50 | 14.749 |
| Jujube | 50 | 14.749 |
| Jujube seed | 30 | 8.850 |
| Gardenia | 30 | 8.850 |
| Poria cocos | 30 | 8.850 |
| Red ginseng | 8 | 2.360 |
| Ecklonia cava extract powder | 21 | 6.195 |
| Total | 339 | 100.000 |

### 1-2. Preparation of Tart Cherry Juice Powder Before Mixing

Tart cherry juice powder was manufactured by uniformly mixing 119 kg (combination percentage of 70% (w/w)) of tart cherry juice powder (produced in Poland), 12.75 kg (combination percentage of 7.5% (w/w)) of fish collagen (produced in Vietnam), 9.35 kg (combination percentage of 5.5% (w/w)) of beet root concentrate powder (produced in Poland), 13.6 kg (combination percentage of 8% (w/w)) of vitamin C (produced in China), 5.1 kg (combination percentage of 3% (w/w)) of natural cherry flavor (domestically produced), and 10.2 kg (combination percentage of 6% (w/w)) of enzymatically modified stevia (See Table 2).

TABLE 2

| | Combination | % by |
|---|---|---|
| | amount (kg) | weight (w/w) |
| Tart cherry juice powder | 119 | 70.00 |
| Fish collagen | 12.75 | 7.50 |
| Beet root concentrate powder | 9.35 | 5.50 |
| Vitamin C | 13.6 | 8.00 |
| Natural cherry flavor | 5.1 | 3.00 |
| Enzymatically modified stevia | 10.2 | 6.00 |
| Total | 170 | 100.00 |

### 1-3. Mixing Process

4000 L of purified water was poured into the pre-sterilized mixing tank in the Embodiment 1-1, and next, 339 kg of the concentrate mixture made in the Embodiment 1-1 and 170 kg of the mixture made in the Embodiment 1-2 were poured into the mixing tank. After 5 that, 4 kg of L-theanine (produced in China) was put and agitated to make 4513 kg of mixture (drink composition) (See Table 3).

TABLE 3

| | Combination | % by |
|---|---|---|
| | amount (kg) | weight (w/w) |
| Siberian ginseng trunk | 70 | 1.551 |
| Wheat | 50 | 1.108 |
| Lotus seed | 50 | 1.108 |
| Jujube | 50 | 1.108 |
| Jujube seed | 30 | 0.665 |
| Gardenia | 30 | 0.665 |
| Poria cocos | 30 | 0.665 |
| Red ginseng | 8 | 0.177 |
| Ecklonia cava extract powder | 21 | 0.465 |
| Tart cherry juice powder | 119 | 2.637 |
| Fish collagen | 12.75 | 0.283 |
| Beet root concentrate powder | 9.35 | 0.207 |
| Vitamin C | 13.6 | 0.301 |
| Natural cherry flavor | 5.1 | 0.113 |
| Enzymatically modified stevia | 10.2 | 0.226 |
| L-theanine | 4 | 0.089 |
| Purified water | 4000 | 88.632 |
| Total | 4513 | 100.000 |

### Embodiment 2. Manufacturing Concentrate Mixture Except Siberian Ginseng Trunk and Red Ginseng

A concentrate mixture was made in the same method as the Embodiment 1-1, but Triticum aestivum (wheat) having the same mass as Eleutherococcus sessiliflorus (Siberian ginseng trunk) and red ginseng was added to make the concentrate mixture.

### Embodiment 3. Manufacturing Concentrate Mixture Except Ecklonia Cava Extract Powder and Gardenia

A concentrate mixture was made in the same method as the Embodiment 1-1, but Triticum aestivum (wheat) having the same mass as Ecklonia cava extract powder and gardenia was added to make the concentrate mixture.

### Embodiment 4. Manufacturing Drink Comprising Blueberry Juice Powder

A mixture (drink composition) was made in the same method as the Embodiment 1, but instead of tart cherry juice powder in the Embodiment 1-1, blueberry juice powder (originated from U.S.A. and made by Haechansol food Co. Ltd., Brix 67) was added. Combination percentages/composition ratios of the made mixture (drink composition) are listed in Table 4.

TABLE 4

| | Combination | % by |
|---|---|---|
| | amount (kg) | weight (w/w) |
| Siberian ginseng trunk | 70 | 1.551 |
| Wheat | 50 | 1.108 |
| Lotus seed | 50 | 1.108 |
| Jujube | 50 | 1.108 |
| Jujube seed | 30 | 0.665 |
| Gardenia | 30 | 0.665 |
| Poria cocos | 30 | 0.665 |
| Red ginseng | 8 | 0.177 |
| Ecklonia cava extract powder | 21 | 0.465 |
| Blueberry juice powder | 119 | 2.637 |
| Fish collagen | 12.75 | 0.283 |
| Beet root concentrate powder | 9.35 | 0.207 |
| Vitamin C | 13.6 | 0.301 |
| Natural cherry flavor | 5.1 | 0.113 |
| Enzymatically modified stevia | 10.2 | 0.226 |
| L-theanine | 4 | 0.089 |
| Purified water | 4000 | 88.632 |
| Total | 4513 | 100.000 |

### Experimental Example 1. Checking Whether Concentrate Mixture Decreases Sleep Latency and Increases Sleep Duration

To check whether the concentrate mixture of the present invention decreases sleep latency and increases sleep duration, the following experiments were carried out.

In detail, 100 ml of the concentrate mixtures made in the Embodiment 1-1, Embodiment 2, and Embodiment 3 were drunk by 10 male and female test subjects every day for 14 days, and then, sleep latency and sleep duration of the test subjects were measured. As the test subjects, 10 men and women who do not have any sleep disorder and difficulties in sleep were chosen. The effectiveness of the sleep induction of the test subjects was measured total three times, that is, before taking the concentrate mixtures, after taking the concentrate mixtures every day for a week before going to bed, and after taking the concentrate mixtures every day for a week before going to bed and next stopping the taking for a week. The measured results are listed in Tables 5 to 10.

TABLE 5

| | Test | 1st | 2nd | 3rd | 4th | 5th | 6th | 7th | 8th | 9th | 10th | 11th | 12th | 13th | 14th | |
|---|---|---|---|---|---|---|---|---|---|---|---|---|---|---|---|---|
| | Subject | day | day | day | day | day | day | day | day | day | day | day | day | day | day | Mean |
| Sleep | No. 1 | 4.6 | 3.9 | 4.3 | 4.4 | 4.6 | 4.2 | 4.4 | 4.3 | 3.9 | 4.3 | 3.8 | 4.4 | 42 | 3.3 | 4.2 |
| induction time | No. 2 | 6.7 | 6.7 | 7.5 | 6.6 | 6.7 | 5.7 | 5.6 | 6.4 | 5.6 | 5.6 | 6.2 | 5.7 | 5.7 | 5.4 | 6.2 |
| under taking | No. 3 | 9.1 | 8.9 | 8.3 | 5.9 | 7.2 | 6.9 | 6.3 | 6.3 | 6.5 | 5.7 | 4.7 | 4.6 | 4.7 | 4.9 | 6.4 |
| mixture of | No. 4 | 8.6 | 8.7 | 8.3 | 8.1 | 7.8 | 8.5 | 6.2 | 6.5 | 5.7 | 4.6 | 5.5 | 4.2 | 4.1 | 3.6 | 6.5 |
| Embodiment | No. 5 | 10.3 | 10.4 | 9.8 | 9.8 | 9.5 | 10.2 | 7.9 | 8.2 | 7.4 | 6.3 | 7.2 | 5.9 | 5.8 | 5.3 | 8.1 |
| 1-1 | No. 6 | 11.3 | 11.4 | 10.3 | 10.3 | 10 | 10.7 | 8.4 | 9.1 | 7.9 | 7.5 | 8.1 | 7.2 | 7.3 | 6.8 | 9.0 |
| | No. 7 | 8.3 | 8.4 | 7.3 | 7.3 | 6.5 | 7.7 | 5.4 | 6.1 | 4.9 | 4.5 | 5.1 | 4.2 | 4.3 | 3.8 | 6.0 |
| | No. 8 | 8.9 | 8.7 | 7.9 | 7.9 | 7.6 | 8.3 | 6.2 | 6.7 | 5.5 | 5.1 | 5.7 | 4.8 | 4.9 | 4.4 | 6.6 |
| | No. 9 | 9.5 | 9.6 | 8.5 | 8.5 | 8.2 | 8.9 | 6.6 | 6.5 | 6.1 | 5.7 | 5.7 | 5.4 | 5.5 | 4.7 | 7.1 |
| | No. 10 | 8.4 | 9.2 | 8.3 | 7.5 | 7.8 | 8.5 | 6.2 | 6.3 | 5.7 | 5.3 | 5.3 | 5.2 | 5.3 | 4.6 | 6.7 |

| | |
|---|---|
| Total mean(min) | 6.7 |

TABLE 6

| | Test | 1st | 2nd | 3rd | 4th | 5th | 6th | 7th | 8th | 9th | 10th | 11th | 12th | 13th | 14th | |
|---|---|---|---|---|---|---|---|---|---|---|---|---|---|---|---|---|
| | Subject | day | day | day | day | day | day | day | day | day | day | day | day | day | day | Mean |
| Sleep | No. 1 | 5.5 | 4.8 | 5.2 | 5.3 | 5.5 | 5.1 | 5.3 | 5.2 | 4.8 | 5.2 | 4.7 | 5.3 | 5.1 | 4.2 | 5.1 |
| induction time | No. 2 | 8.2 | 8.2 | 8.5 | 8.1 | 8.2 | 7.2 | 7.1 | 7.9 | 7.1 | 7.1 | 7.5 | 7.2 | 7.2 | 6.9 | 7.6 |
| under taking | No. 3 | 11.3 | 11.1 | 10.5 | 8.1 | 9.2 | 9.1 | 8.5 | 8.5 | 8.7 | 8.2 | 7.2 | 6.8 | 7.2 | 7.1 | 8.7 |
| mixture of | No. 4 | 11.6 | 11.7 | 11.3 | 11.1 | 10.8 | 11.5 | 9.2 | 9.5 | 8.7 | 7.6 | 8.5 | 7.2 | 7.1 | 6.6 | 9.5 |
| Embodiment | No. 5 | 11.3 | 11.4 | 10.8 | 10.8 | 10.5 | 11.2 | 8.9 | 9.2 | 8.4 | 7.3 | 8.2 | 6.9 | 6.8 | 6.3 | 9.1 |
| 2 | No. 6 | 12.8 | 12.9 | 11.8 | 11.8 | 11.5 | 12.2 | 9.9 | 10.6 | 9.4 | 9 | 9.6 | 8.7 | 8.8 | 8.3 | 10.5 |
| | No. 7 | 11.3 | 11.4 | 10.3 | 10.3 | 10 | 10.7 | 8.4 | 9.1 | 7.9 | 7.5 | 8.1 | 7.2 | 7.3 | 6.8 | 9.0 |
| | No. 8 | 10.6 | 10.7 | 9.6 | 9.6 | 9.3 | 10 | 7.7 | 8.4 | 7.2 | 6.8 | 7.4 | 6.5 | 6.6 | 6.1 | 8.3 |
| | No. 9 | 10.2 | 10.3 | 9.2 | 9.2 | 8.9 | 9.6 | 7.3 | 7.2 | 6.8 | 6.4 | 6.4 | 6.1 | 6.2 | 5.7 | 7.8 |
| | No. 10 | 8.3 | 9.1 | 8.2 | 7.4 | 7.7 | 8.4 | 6.1 | 6.2 | 5.6 | 5.2 | 5.2 | 4.9 | 5.2 | 4.5 | 6.6 |

| | |
|---|---|
| Total mean(min) | 8.2 |

TABLE 7

| | Test | 1st | 2nd | 3rd | 4th | 5th | 6th | 7th | 8th | 9th | 10th | 11th | 12th | 13th | 14th | |
|---|---|---|---|---|---|---|---|---|---|---|---|---|---|---|---|---|
| | Subject | day | day | day | day | day | day | day | day | day | day | day | day | day | day | Mean |
| Sleep | No. 1 | 10.1 | 9 | 9 | 9.0 | 10.1 | 9.7 | 9.9 | 9.8 | 9.4 | 9.8 | 9.3 | 9.9 | 9.7 | 9.8 | 9.7 |
| induction time | No. 2 | 15.5 | 15.5 | 15.8 | 15.4 | 15.5 | 14.5 | 14.4 | 15.2 | 14.4 | 14.4 | 14.8 | 14.5 | 14.5 | 14.2 | 14.9 |
| under taking | No. 3 | 19.6 | 19.4 | 18.8 | 16.4 | 17.5 | 17.4 | 16.8 | 16.8 | 17 | 16.5 | 15.5 | 15.1 | 15.5 | 15.4 | 17.0 |
| mixture of | No. 4 | 18.6 | 18.7 | 18.3 | 18.1 | 17.8 | 18.5 | 16.2 | 16.5 | 15.7 | 14.6 | 15.5 | 14.2 | 14.1 | 13.6 | 16.5 |
| Embodiment | No. 5 | 19.5 | 19.6 | 19 | 19 | 18.7 | 19.4 | 17.1 | 17.4 | 16.6 | 15.5 | 16.4 | 15.1 | 15 | 14.5 | 17.3 |
| 3 | No. 6 | 20.6 | 20.7 | 19.6 | 19.6 | 19.3 | 20 | 17.7 | 18.4 | 17.2 | 16.8 | 17.4 | 16.5 | 16.6 | 16.1 | 18.3 |
| | No. 7 | 19.1 | 19.2 | 18.1 | 18.1 | 17.8 | 18.5 | 16.2 | 16.9 | 15.7 | 15.3 | 15.9 | 15 | 15.1 | 14.6 | 16.8 |
| | No. 8 | 19.8 | 19.9 | 18.8 | 18.8 | 18.5 | 19.2 | 16.9 | 17.6 | 16.4 | 16 | 16.6 | 157. | 15.8 | 15.3 | 17.5 |
| | No. 9 | 18.3 | 18.4 | 17.3 | 17.3 | 17 | 17.7 | 15.4 | 15.3 | 14.9 | 14.5 | 14.5 | 14.2 | 14.3 | 13.8 | 15.9 |
| | No. 10 | 14.9 | 15.7 | 14.8 | 14 | 14.3 | 15 | 12.7 | 12.8 | 12.2 | 11.8 | 11.8 | 11.5 | 11.8 | 11.1 | 13.2 |

| | |
|---|---|
| Total mean(min) | 15.7 |

TABLE 8

| | Test | 1st | 2nd | 3rd | 4th | 5th | 6th | 7th | 8th | 9th | 10th | 11th | 12th | 13th | 14th | |
|---|---|---|---|---|---|---|---|---|---|---|---|---|---|---|---|---|
| | Subject | day | day | day | day | day | day | day | day | day | day | day | day | day | day | Mean |
| Sleep time | No. 1 | 5.7 | 5.7 | 5.9 | 5.9 | 6 | 6.1 | 6 | 6 | 6.1 | 6.1 | 6.1 | 6 | 6 | 6.1 | 6 |
| under taking | No. 2 | 5.6 | 5.8 | 5.8 | 5.8 | 5.9 | 6 | 5.9 | 5.9 | 6 | 6 | 6 | 5.9 | 5.9 | 6 | 5.9 |
| mixture of | No. 3 | 5.5 | 5.5 | 5.7 | 5.7 | 6 | 5.9 | 5.8 | 5.8 | 5.9 | 5.9 | 5.9 | 5.8 | 5.9 | 5.9 | 5.8 |
| Embodiment | No. 4 | 5.5 | 5.5 | 5.7 | 5.7 | 5.9 | 5.9 | 5.8 | 5.8 | 5.9 | 5.9 | 5.9 | 5.8 | 5.9 | 5.9 | 5.8 |
| 1-1 | No. 5 | 5.6 | 5.6 | 5.8 | 5.8 | 5.9 | 6 | 5.9 | 5.9 | 6 | 6 | 6 | 5.9 | 6 | 6.1 | 5.9 |
| | No. 6 | 5.5 | 5.5 | 5.7 | 5.7 | 5.8 | 5.9 | 5.8 | 5.8 | 5.9 | 5.9 | 5.9 | 5.8 | 5.9 | 6 | 5.8 |
| | No. 7 | 5.4 | 5.4 | 5.6 | 5.6 | 5.7 | 5.8 | 5.7 | 5.7 | 5.8 | 6 | 6 | 5.9 | 5.9 | 6.1 | 5.8 |
| | No. 8 | 5.3 | 5.3 | 5.5 | 5.5 | 5.6 | 5.7 | 5.6 | 5.8 | 5.9 | 5.9 | 5.9 | 5.9 | 5.9 | 6 | 5.7 |
| | No. 9 | 5.4 | 5.4 | 5.6 | 5.6 | 5.7 | 5.8 | 5.7 | 5.9 | 6 | 6 | 6 | 5.9 | 5.9 | 6.1 | 5.8 |
| | No. 10 | 5.2 | 5.2 | 5.4 | 5.4 | 5.5 | 5.6 | 5.5 | 5.7 | 5.8 | 5.9 | 5.9 | 5.8 | 5.9 | 6 | 5.6 |

| | |
|---|---|
| Total mean(min) | 5.8 |

TABLE 9

| | Test | 1st | 2nd | 3rd | 4th | 5th | 6th | 7th | 8th | 9th | 10th | 11th | 12th | 13th | 14th | |
|---|---|---|---|---|---|---|---|---|---|---|---|---|---|---|---|---|
| | Subject | day | day | day | day | day | day | day | day | day | day | day | day | day | day | Mean |
| Sleep time | No. 1 | 4.1 | 4.1 | 4.3 | 4.3 | 4.2 | 4.3 | 4.2 | 4.4 | 4.3 | 4.3 | 4.5 | 4.4 | 4.4 | 4.5 | 4.3 |
| under taking | No. 2 | 4 | 4.2 | 4.2 | 4.2 | 4.1 | 4.2 | 4.1 | 4.3 | 4.2 | 4.2 | 4.4 | 4.3 | 4.3 | 4.4 | 4.2 |
| mixture of | No. 3 | 3.9 | 3.9 | 4.1 | 4.1 | 4.2 | 4.1 | 4 | 4.2 | 4.1 | 4.1 | 4.3 | 4.2 | 4.3 | 4.3 | 4.1 |
| Embodiment | No. 4 | 3.9 | 3.9 | 4.1 | 4.1 | 4.1 | 4.1 | 4 | 4.2 | 4.1 | 4.1 | 4.3 | 4.2 | 4.3 | 4.3 | 4.1 |
| 2 | No. 5 | 4 | 4 | 4.2 | 4.2 | 4.1 | 4.2 | 4.1 | 4.3 | 4.2 | 4.2 | 4.4 | 4.3 | 4.4 | 4.5 | 4.2 |
| | No. 6 | 3.9 | 3.9 | 4.1 | 4.1 | 4 | 4.1 | 4 | 4.2 | 4.1 | 4.1 | 4.3 | 4.2 | 4.3 | 4.4 | 4.1 |
| | No. 7 | 3.8 | 3.8 | 4 | 4 | 3.9 | 4 | 3.9 | 4.1 | 4 | 4.2 | 4.4 | 4.3 | 4.3 | 4.5 | 4.1 |
| | No. 8 | 3.7 | 3.7 | 3.9 | 3.9 | 3.8 | 3.9 | 3.8 | 4.2 | 4.1 | 4.1 | 4.3 | 4.3 | 4.3 | 4.4 | 4.0 |
| | No. 9 | 3.8 | 3.8 | 4 | 4 | 3.9 | 4 | 3.9 | 4.3 | 4.2 | 4.2 | 4.4 | 4.3 | 4.3 | 4.5 | 4.1 |
| | No. 10 | 3.6 | 3.6 | 3.8 | 3.8 | 3.7 | 3.8 | 3.7 | 4.1 | 4 | 4.1 | 4.3 | 4.2 | 4.3 | 4.4 | 4.0 |

| | |
|---|---|
| Total mean(min) | 4.1 |

TABLE 10

| | Test | 1st | 2nd | 3rd | 4th | 5th | 6th | 7th | 8th | 9th | 10th | 11th | 12th | 13th | 14th | |
|---|---|---|---|---|---|---|---|---|---|---|---|---|---|---|---|---|
| | Subject | day | day | day | day | day | day | day | day | day | day | day | day | day | day | Mean |
| Sleep time | No. 1 | 5.2 | 5.2 | 5.4 | 5.4 | 5.3 | 5.4 | 5.3 | 5.5 | 5.4 | 5.4 | 5.4 | 5.3 | 5.3 | 5.4 | 5.4 |
| under taking | No. 2 | 5.1 | 5.3 | 5.3 | 5.3 | 5.2 | 5.3 | 5.2 | 5.4 | 5.3 | 5.3 | 5.3 | 5.2 | 5.2 | 5.3 | 5.3 |
| mixture of | No. 3 | 5 | 5 | 5.2 | 5.2 | 5.3 | 5.2 | 5.1 | 5.3 | 5.2 | 5.2 | 5.2 | 5.1 | 5.2 | 5.2 | 5.2 |
| Embodiment | No. 4 | 5 | 5. | 5.2 | 5.2 | 5.2 | 5.2 | 5.1 | 5.3 | 5.2 | 5.2 | 5.2 | 5.1 | 5.2 | 5.2 | 5.2 |
| 3 | No. 5 | 5.1 | 5.1 | 5.3 | 5.3 | 5.2 | 5.3 | 5.2 | 5.4 | 5.3 | 5.3 | 5.3 | 5.2 | 5.3 | 5.4 | 5.3 |
| | No. 6 | 5 | 5 | 5.2 | 5.2 | 5.1 | 5.2 | 5.1 | 5.3 | 5.2 | 5.2 | 5.2 | 5.1 | 5.2 | 5.3 | 5.2 |
| | No. 7 | 4.9 | 4.9 | 5.1 | 5.1 | 5 | 5.1 | 5 | 5.2 | 5.1 | 5.3 | 5.3 | 5.2 | 5.2 | 5.4 | 5.1 |
| | No. 8 | 4.8 | 4.8 | 5 | 5 | 4.9 | 5 | 4.9 | 5.3 | 5.2 | 5.2 | 5.2 | 5.2 | 5.2 | 5.3 | 5.1 |
| | No. 9 | 4.9 | 4.9 | 5.1 | 5.1 | 5 | 5.1 | 5 | 5.4 | 5.3 | 5.3 | 5.3 | 5.2 | 5.2 | 5.4 | 5.2 |
| | No. 10 | 4.7 | 4.7 | 4.9 | 4.9 | 4.8 | 4.9 | 4.8 | 5.2 | 5.1 | 5.2 | 5.2 | 5.1 | 5.2 | 5.3 | 5.0 |

| | |
|---|---|
| Total mean(min) | 5.2 |

As a result, as shown in Tables 5 to 10, it was checked that the concentrate mixture made according to the Embodiment 1-1 had significant efficacy of sleep disorder improvement.

In detail, in the case of the Embodiment 2 in which Siberian ginseng trunk and red ginseng are not contained, total mean sleep induction time is 8.2 minutes, which is shorter by about 7.5 minutes than that of the Embodiment 3 but slower by about 1.5 minutes than that of the Embodiment 1-1. Further, the mean sleep duration of the Embodiment 2 is 4.1 hours, which is the shortest sleep duration among the three samples, and the Embodiment 3 is the longest sleep induction time among the three samples, but the sleep duration thereof is 5.2 hours, which is longer than that of the Embodiment 2. In the case of the Embodiment 1-1, mean sleep induction time is 6.7 minutes and mean sleep duration is 5.8 hours, from which it can be appreciated that the sleep latency decreases and the sleep duration increases. Accordingly, it can be appreciated that when the concentrate mixture of the Embodiment 1-1 is taken, sleep induction and sleep disorder improvement are excellent.

### Experimental Example 2. Checking Whether Concentrate Mixture Improves the Pittsburgh Sleep Quality Index

To evaluate the sleep quality improvement efficacy of the concentrate mixture of the present invention, after the concentrate mixture made in the Embodiment 1-1 was taken, the Pittsburgh sleep quality index was measured by the following method.

Sleep scales as generally used include the Pittsburgh sleep quality index (PSQI), insomnia severity index (ISI), Epworth sleepiness scale (ESS), etc., but to check general sleep efficiency or various aspects of sleep, the Pittsburgh sleep quality index was used in the present invention. In Korea, Korean PSQI (PSQI-K) were approved in reliability and validity, and accordingly, using the Korean PSQI (PSQI-K), the following tests were carried out (See Journal of Convergence for Information Technology Vol. 10. No. 11, pp. 148-155, 2020.).

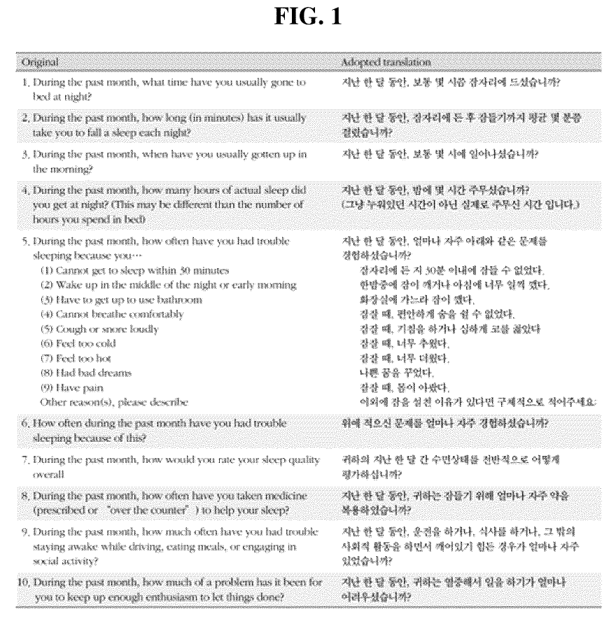

First, 20 men and women test subjects having difficulties in sleep were chosen. In the same manner as in the Experimental example 1, next, 100 ml of the concentrate mixture made in the Embodiment 1-1 was drunk by the 20 test subjects every day for 14 days. To check sleep quality improvement, the effectiveness of the sleep induction of the test subjects was measured total three times, that is, before taking the concentrate mixture, after taking the concentrate mixture every day for a week before going to bed, and after taking the concentrate mixture every day for a week before going to bed and next stopping the taking for a week. The measured results are listed in FIGS. 1 to 4.

Further, questions of the Pittsburgh sleep quality index used for the tests are listed in FIG. 1, and in detail, there are 18 questions for seven components, for example, sleep quality (one question), sleep latency (two questions), sleep duration (one question), sleep efficiency (one question), sleep disturbance (nine questions), sleep medication use (one question), and daytime dysfunction (two questions). With a scale of 0 to 3 according to each component, the scale 0 represents no difficulty and the scale 21 severe difficulty, and if the score is over 5, it means bad sleep quality.

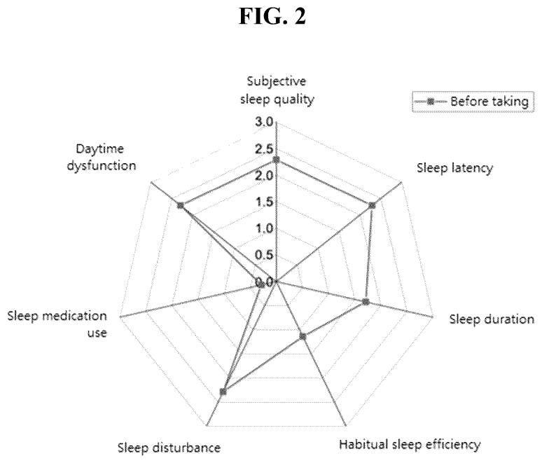

FIG. 2 shows a sleep quality evaluation (primary evaluation) result for one month before the concentrate mixture of the present invention is taken. As shown in FIG. 2, five components among the seven components show high scores more than 1.5, and before taking the concentrate mixture, especially, the score of the Pittsburgh sleep quality index is 12.29, which exceeds the score 5 as suggested by Buysse et al., 1989.

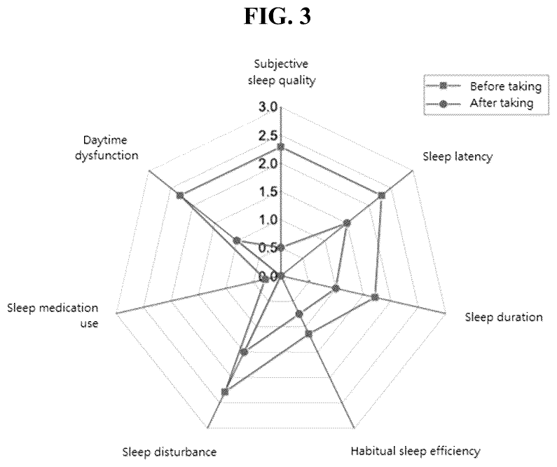

FIG. 3 shows a sleep quality evaluation (secondary evaluation) result after the primary evaluation of the Pittsburgh sleep quality index is performed and the concentrate mixture of the present invention is then taken every day for a week. The score of the secondary evaluation is 6.25, which is half of the score 12.29 of the primary evaluation. Referring to the seven components, it can be checked that the existing score 2.29 of the subjective sleep quality is changed to the score 0.5, which shows remarkable sleep quality improvement. Further, it is checked that the overall sleep quality index of the 7 components is reduced and the scores of the sleep duration and sleep latency are also reduced almost by half, which proves excellent efficacy of the concentrate mixture of the present invention.

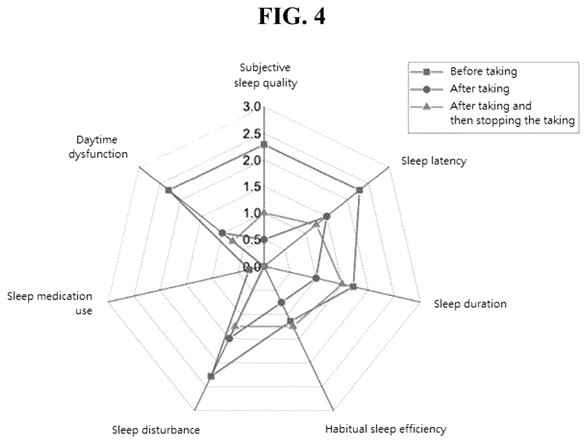

FIG. 4 shows a sleep quality evaluation result for a week after a week of the secondary evaluation of the Pittsburgh sleep quality index. As a result, it is checked that the score 6.25 of the secondary evaluation is changed to the score 7, and accordingly, it is appreciated that the sleep quality is improved specifically through the concentrate mixture of the present invention. Referring to the seven components, it can be checked that the habitual sleep efficiency decreases as the secondary evaluation is carried out after the primary evaluation has been completed, but after taking is stopped, it can be checked that the habitual sleep efficiency is returned to original habit. Further, it is checked that the index scores of the subjective sleep quality and the sleep duration after taking is stopped are more increased than those after the concentrate mixture is taken, which are similar to the index scores before the concentrated mixture is taken.

In summary, the sleep quality evaluation results for one month before the concentrate mixture of the present invention is taken, after taking for a week, and for a week after the taking is stopped show the scores 12.29, 6.25, and 7, and accordingly, it is appreciated that the evaluation index score before taking has two times higher than that after taking, which means that the concentrate mixture of the present invention remarkably improves the sleep quality.

### Experimental Example 3. Sensory Evaluation Test for Drink Made

Using the mixtures (drink compositions) made in the methods of the Embodiments 1 to 4, the following sensory evaluation tests were carried out.

As people are aged, the number of taste buds drastically decreases and taste cells are degenerated so that the ability to taste become reduced. If a person is in his or her late fifties, he or she has low sensitivity to bitter and sweet tastes as well as smell, and accordingly, 10 adults aged 25 to 45 years were chosen as an evaluation group to evaluate the preference of the drink compositions.

In detail, before the sensory evaluation tests, purpose of test, evaluation items, and scale use methods were explained to the evaluation group, and next, the drink compositions were made in the methods of the Embodiments 1 to 4. The drink compositions were put at a room temperature an hour ago before the sensory evaluation test, and next, 100 mL of drink compositions were drunk by them. With constant time intervals among the sensory evaluation tests, the preference for outer appearances (colors), flavors, sweet tastes, bitter tastes, senses of drinking, and overall preference was evaluated using nine-point scale. The evaluation results are listed in Table 11.

TABLE 11

| | Outer | | | | | |
|---|---|---|---|---|---|---|
| | appearance | | Sweet | Bitter | Sense of | Overall |
| | (color) | Flavor | taste | taste | drinking | preference |
| Drink | 7.4 | 7.9 | 8.2 | 7.8 | 8.1 | 7.88 |
| comprising tart | | | | | | |
| cherry juice | | | | | | |
| powder | | | | | | |
| Drink | 6.5 | 4 | 7.4 | 6.5 | 5.7 | 6.02 |
| comprising | | | | | | |
| blueberry juice | | | | | | |
| powder | | | | | | |

As appreciated from Table 11, if the drink composition comprising tart cherry juice powder made in the method of the Embodiment 1 is drunk, it has higher outer appearance (color), flavor, sweet taste, sense of drinking, and overall preference than the drink composition comprising blueberry juice powder made in the method of the Embodiment 4. If the tart cherry juice powder is used, it has relatively higher bitter taste strength than the blueberry juice powder, but it is appreciated that the flavor and the sweet taste of the tart cherry juice powder are mixed with each other to improve all of the outer appearance (color), flavor, sweet taste, sense of drinking, and overall preference of the drink composition.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention. For example, the parts expressed in a singular form may be dispersedly provided, and in the same manner as above, the parts dispersed may be combined with each other.

Therefore, the present invention is not to be restricted by the embodiments as mentioned above. It is therefore intended that the scope of the invention be limited not by this detailed description, but rather by the claims appended hereto.

### INDUSTRIAL APPLICABILITY

The present invention relates to the sleep induction or sleep disorder improvement composition comprising natural product-derived ingredients as active ingredients, thereby being useful in sleep induction or sleep disorder improvement.

1. A food composition for sleep induction or sleep disorder improvement, the food composition comprising one or more active ingredients selected from the group consisting of Siberian ginseng trunk, wheat, lotus seed, jujube, jujube seed, gardenia, Poria cocos, red ginseng, Ecklonia cava extract powder, tart cherry juice powder, fish collagen, beet root concentrate powder, vitamin C, enzymatically modified stevia, and L-theanine.

2. The food composition according to claim 1, wherein the Siberian ginseng trunk is present in an amount of 0.5 to 3 parts by weight in the total weight thereof as 100 parts by weight.

3. The food composition according to claim 1, wherein the sleep induction allows sleep latency to decrease or allows sleep duration to increase.

4. The food composition according to claim 1, wherein the sleep disorder comprises one or more disorders selected from the group consisting of sleep latency disorder, deep sleep disorder, awakening on night sleep, early morning awakening, insomnia, nightmare, sleepwalking, narcolepsy, unusual behaviors during sleep, hypersomnia, sleep seizure, sleep-disordered breathing, sleep apnea, circadian rhythm sleep disorders, parasomnia, restless legs syndrome, and periodic limb movement disorder.

5. A health functional food composition for sleep induction or sleep disorder improvement, the health functional food composition comprising one or more active ingredients selected from the group consisting of Siberian ginseng trunk, wheat, lotus seed, jujube, jujube seed, gardenia, Poria cocos, red ginseng, Ecklonia cava extract powder, tart cherry juice powder, fish collagen, beet root concentrate powder, vitamin C, enzymatically modified stevia, and L-theanine.

6. The health functional food composition according to claim 5, wherein the Siberian ginseng trunk is present in an amount of 0.5 to 3 parts by weight in the total weight thereof as 100 parts by weight.

7. The health functional food composition according to claim 5, wherein the sleep induction allows sleep latency to decrease or allows sleep duration to increase.

8. The health functional food composition according to claim 5, wherein the sleep disorder comprises one or more disorders selected from the group consisting of sleep latency disorder, deep sleep disorder, awakening on night sleep, early morning awakening, insomnia, nightmare, sleepwalking, narcolepsy, unusual behaviors during sleep, hypersomnia, sleep seizure, sleep-disordered breathing, sleep apnea, circadian rhythm sleep disorders, parasomnia, restless legs syndrome, and periodic limb movement disorder.

9. A medicinal composition for sleep disorder prevention or treatment, comprising one or more active ingredients selected from the group consisting of Siberian ginseng trunk, wheat, lotus seed, jujube, jujube seed, gardenia, Poria cocos, red ginseng, and Ecklonia cava extract powder.

10. The medicinal composition according to claim 9, wherein the Siberian ginseng trunk is present in an amount of 0.5 to 3 parts by weight in the total weight thereof as 100 parts by weight.

11. The medicinal composition according to claim 9, wherein the sleep induction allows sleep latency to decrease or allows sleep duration to increase.
